(19) United States
(12) Patent Application Publication
(10) Pub. No.: US 2025/0152388 A1
(43) Pub. Date: May 15, 2025

(54) NOVEL ENHANCED STENT MAKING METHODS & APPLICATIONS OF THE SAME WITHIN VASCULAR SYSTEM NEEDING THE SAME

(71) Applicant: M4D, LLC, Lake Forest, CA (US)

(72) Inventor: Tuan Ngo, Lake Forest, CA (US)

(21) Appl. No.: 18/945,906

(22) Filed: Nov. 13, 2024

Related U.S. Application Data
(60) Provisional application No. 63/598,199, filed on Nov. 13, 2023.

(51) Int. Cl. A61F 2/89; B21D 39/04

(52) U.S. Cl. CPC A61F 2/89; B21D 39/048; A61F 2240/001

(57) ABSTRACT

Improved stenting systems allow better placement without issues and placement, for example in the carotid arteries more facilely and rely during deployment than prior art.

### CROSS-REFERENCE TO RELATED APPLICATIONS

The present application claims priority to U.S. Provisional Application No. 63/598,199, Filed Nov. 13, 2024, the contents of which are incorporated herein by reference in their entirety.

### FIELD OF THE INVENTION

The field of the invention is compositions, devices, and methods of in devices, especially as it relates to making vessels patent, be that by restoring flow channels, surgical removal of a thrombus or other obstructions or delivery of target therapies within a subject blood vessel.

### BACKGROUND

The background description includes information that may be useful in understanding the present invention. It is not an admission that any of the information provided herein is prior art or relevant to the presently claimed invention, or that any publication specifically or implicitly referenced is prior art.

During thrombectomy with antegrade blood flow, an engaged emboli can become dislodged from the mechanical device. Blood flow has a water-hammer effect on the emboli which can break pieces of the emboli off or entirely dislodge the emboli from the capturing device.

Thus, there is still a need for improved devices to open vessels and methods of thrombectomy devices, and especially neurovascular thrombectomy devices.

### SUMMARY OF THE INVENTION

The present inventive subject matter is drawn to compositions, devices, and methods of varying either the density and main diameter that is sized for vessel diameters of the internal carotid, common carotid Basilar or vertebral arteries, inter alia.

Also the distal tip can be configured with closed end or porous end. The distal tip can be closed with a laser cut configuration and/or covered with PTFE, or a braid of polymer or metal.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 provides an exemplary illustration of the inventive subject matter, depicting a stenting device that can be adjusted in flexibility;

FIG. 2 provides an exemplary illustration of the inventive subject matter, depicting a stenting device that can be adjusted in flexibility;

FIG. 3 provides an exemplary illustration of the inventive subject matter, depicting a stenting device that can be adjusted in flexibility;

FIG. 4 provides an exemplary illustration of the inventive subject matter, depicting a stenting device that can be adjusted in flexibility;

FIG. 5 provides an exemplary illustration of the inventive subject matter, depicting a stenting device that can be adjusted in flexibility;

FIG. 6 provides an exemplary illustration of the inventive subject matter, depicting a stenting device that can be adjusted in flexibility;

FIG. 7 provides an exemplary illustration of the inventive subject matter, depicting a stenting device that can be adjusted in flexibility;

FIG. 8 provides an exemplary illustration of the inventive subject matter, depicting a stenting device that can be adjusted in flexibility;

FIG. 9 provides an exemplary illustration of the inventive subject matter, depicting a stenting device that can be adjusted in flexibility;

FIG. 10 provides an exemplary illustration of the inventive subject matter, depicting a stenting device that can be adjusted in flexibility;

FIG. 11 provides an exemplary illustration of the inventive subject matter, depicting a stenting device in this case a flow diverter that can be adjusted in flexibility;

### DETAILED DESCRIPTION

The inventors have discovered that patency restoration devices with improved implantation characteristics, and especially with improved access, placement and vessel improvement characteristics and/or embolus removed/retention can be prepared by inventive methods for delivering stents having different densities within the bodies of the stents and their crowns, distal or proximate ends.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed. As used in the description herein and throughout the claims that follow, the meaning of “a, an,” and “the” includes plural reference unless the context clearly dictates otherwise. Also, as used in the description herein, the meaning of “in” includes “in” and “on” unless the context clearly dictates otherwise.

Moreover, as used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

In one especially preferred aspect of the inventive subject matter MORE DENSITY IN MIDDLE THAN FLEXIBLE ENDS IS SHOWN, for Carotid usage.

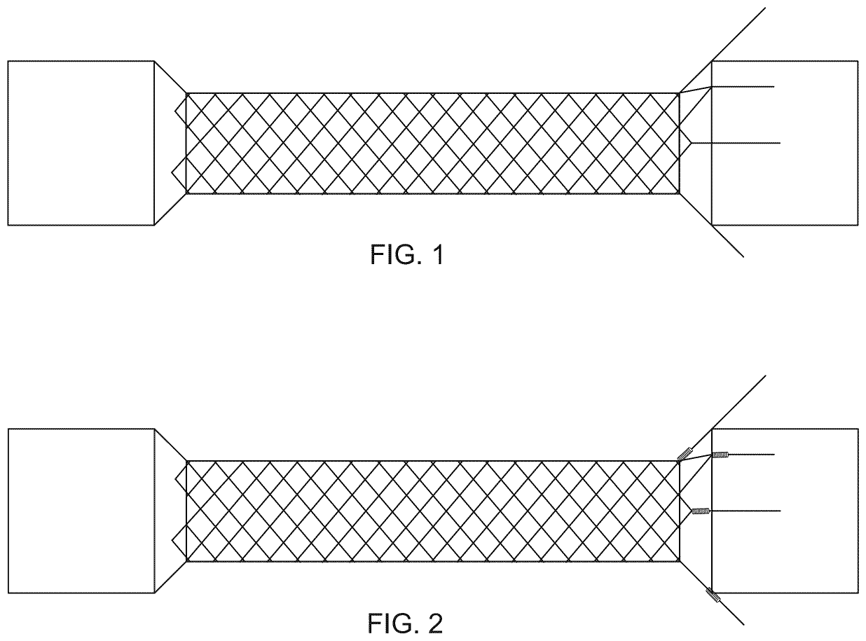

FIG. 1 provides an exemplary illustration of the inventive subject matter, depicting a stenting device that can be adjusted in flexibility;

FIG. 2 provides an exemplary illustration of the inventive subject matter, depicting a stenting device that can be adjusted in flexibility;

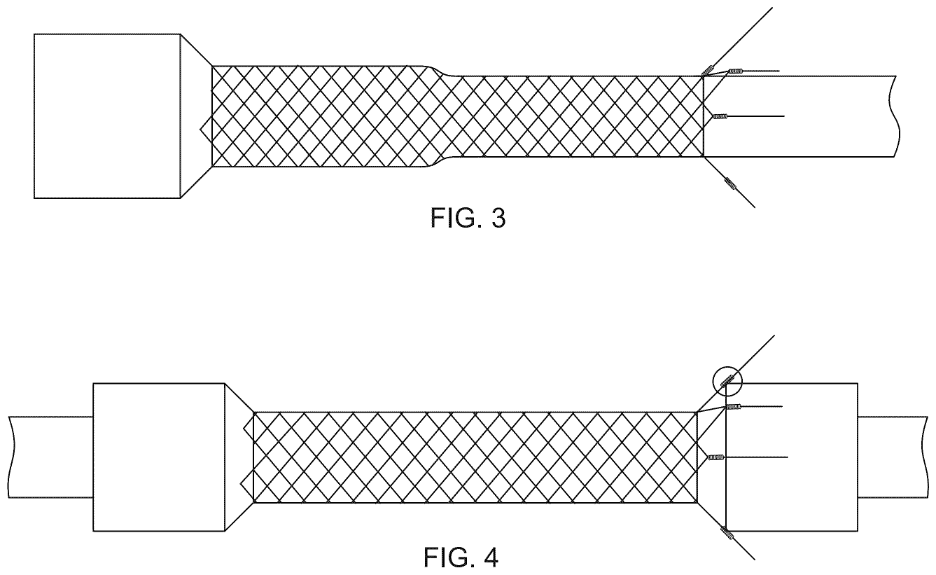

FIG. 3 provides an exemplary illustration of the inventive subject matter, depicting a stenting device that can be adjusted in flexibility;

FIG. 4 provides an exemplary illustration of the inventive subject matter, depicting a stenting device that can be adjusted in flexibility;

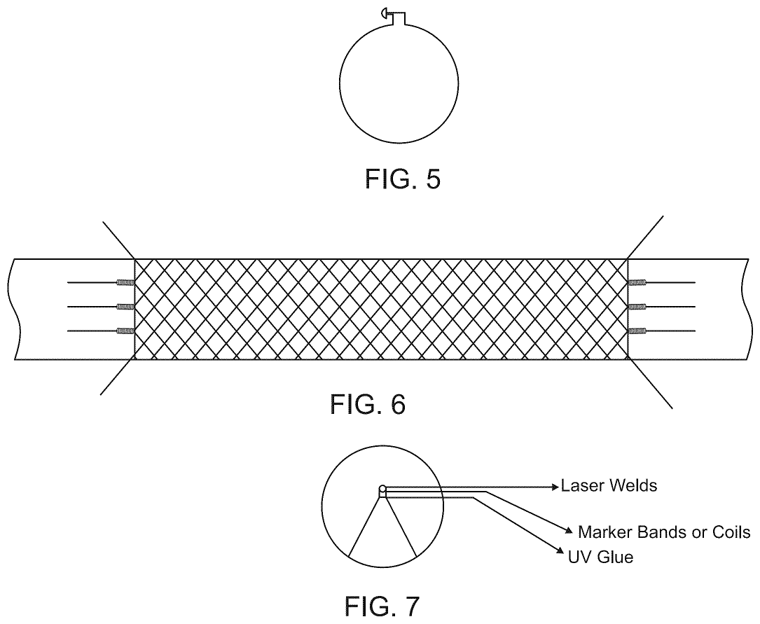

FIG. 5 provides an exemplary illustration of the inventive subject matter, depicting a stenting device that can be adjusted in flexibility;

FIG. 6 provides an exemplary illustration of the inventive subject matter, depicting a stenting device that can be adjusted in flexibility;

FIG. 7 provides an exemplary illustration of the inventive subject matter, depicting a stenting device that can be adjusted in flexibility;

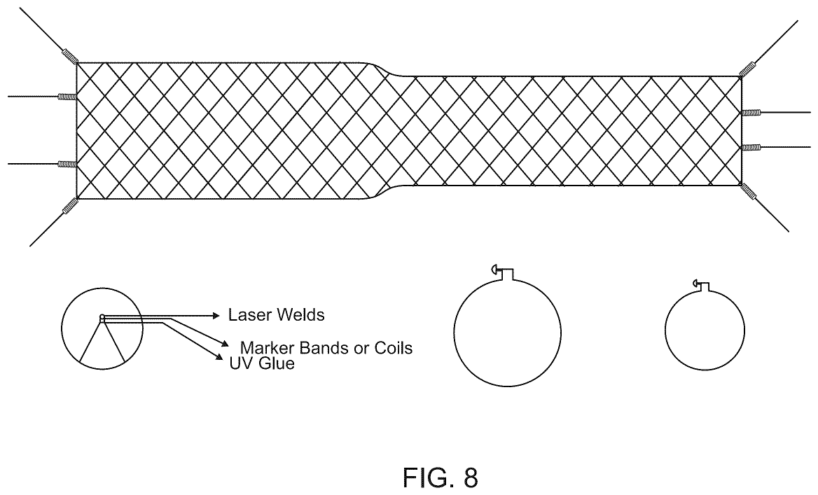

FIG. 8 provides an exemplary illustration of the inventive subject matter, depicting a stenting device that can be adjusted in flexibility;

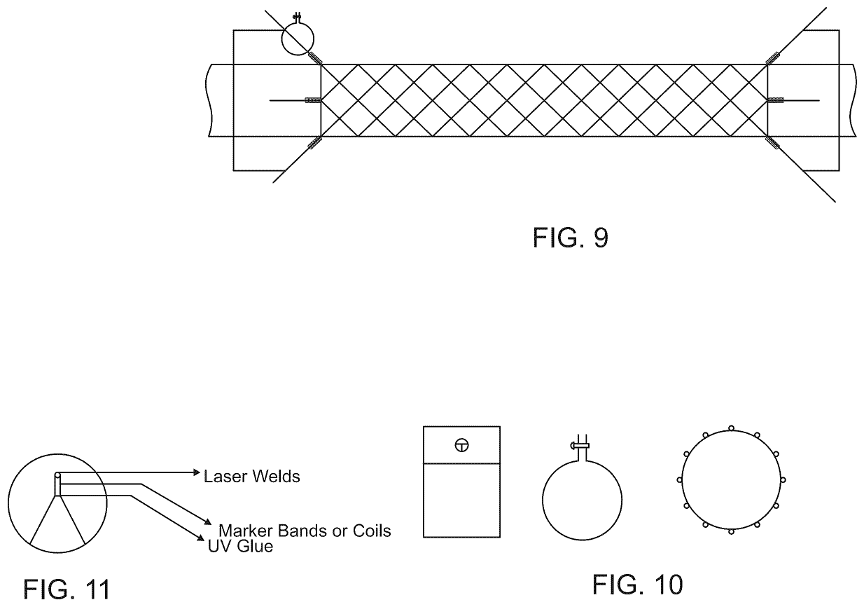

FIG. 9 provides an exemplary illustration of the inventive subject matter, depicting a stenting device that can be adjusted in flexibility;

FIG. 10 provides an exemplary illustration of the inventive subject matter, depicting a stenting device that can be adjusted in flexibility;

FIG. 11 provides an exemplary illustration of the inventive subject matter, depicting a stenting device in this case a flow diverter that can be adjusted in flexibility;

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C, and N, the text should be interpreted as requiring only one element from the group, not A plus N, or B plus N, The present inventor takes all of the empirical knowledge and data from U.S. Pat. No. 9,808,359 (expressly incorporated herein along with all references and publications disclosed therein and attached as Appendix herein), which clinically defines and calibrates vasospasm generally, and applies it to the radial access world.

Further, expressly incorporated are US Letters Patents Numbers 11,768,676; 9,566,071; 9,566,072; 10,398,877; 4,733,665; PCT/US2015/000117; and, PCT/US20/45557. While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

“STENT” is defined as any metallic, polymeric or other materials based miniature vascular interventional cage being able to be sized under at least about 3 to 8 French Catheter scale, or any pieces of the same used for flow-diversion, thrombectomy, and/or PALMAZ® STENT TYPE OF OPEN MESH TUBE meaning it opens a vessel holding the same open to restore vessel patency toward normal blood flow, according to U.S. Pat. No. 4,733,665. The stent may be hand-woven from wire or machined, for example on a STEEGER USA Model Number HS80/48-2021-IMC7K brand of machine which can run any numbers of wires for example 8, 24, 48, 32, 16 and 64 according to preferred embodiments.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

A processor may be provided by one or more processors including, for example, one or more of a single core or multi-core processor (e.g., AMD Phenom II X2, Intel Core Duo, AMD Phenom II X4, Intel Core i5, Intel Core I & Extreme Edition 980X, or Intel Xeon E7-2820).

An I/O mechanism may include a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), an accelerometer, a microphone, a cellular radio frequency antenna, and a network interface device (e.g., a network interface card (NIC), Wi-Fi card, cellular modem, data jack, Ethernet port, modem jack, HDMI port, mini-HDMI port, USB port), touchscreen (e.g., CRT, LCD, LED, AMOLED, Super AMOLED), pointing device, trackpad, light (e.g., LED), light/image projection device, or a combination thereof.

Memory according to the invention refers to a non-transitory memory which is provided by one or more tangible devices which preferably include one or more machine-readable medium on which is stored one or more sets of instructions (e.g., software) embodying any one or more of the methodologies or functions described herein. The software may also reside, completely or at least partially, within the main memory, processor, or both during execution thereof by a computer within system, the main memory and the processor also constituting machine-readable media. The software may further be transmitted or received over a network via the network interface device.

While the machine-readable medium can in an exemplary embodiment be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present invention. Memory may be, for example, one or more of a hard disk drive, solid state drive (SSD), an optical disc, flash memory, zip disk, tape drive, “cloud” storage location, or a combination thereof. In certain embodiments, a device of the invention includes a tangible, non-transitory computer readable medium for memory. Exemplary devices for use as memory include semiconductor memory devices, (e.g., EPROM, EEPROM, solid state drive (SSD), and flash memory devices e.g., SD, micro SD, SDXC, SDIO, SDHC cards); magnetic disks, (e.g., internal hard disks or removable disks); and optical disks (e.g., CD and DVD disks).

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A Stent which is comprised of, in combination:
at least a first density region, extending from crowns at first end along the length of the stent to another;
second density region, wherein the density is respectively defined by a frequency of emplaced wires or struts between the first and second sets of crowns.

2. The stent of claim 1, being targeted for emplacement within the carotid artery.

3. The stent of claim 2, comprised of: 16 wires at 10 mm×40 mm, wherein the end structures and wire density are programed into instructions and/or hand woven or machined by a STEEGER USA Model Number HS80/48-2021-IMC7K brand of machine, in whole or in part, and having 2 bands round a mandrel before hitting.

4. The stent of claim 3, being comprised of 20 at 10 mm×40 mm, wherein the end structures and wire density are programed into instructions and/or hand woven or machined by a STEEGER USA Model Number HS80/48-2021-IMC7K brand of machine in whole or in part, further comprising laser welds, marker bands or coils and UV glue.

5. The stent of claim 4, further comprised of an improved step down (reverse frusto-conical or PINCHUK TYPES of stent) having wires at 8 mm, 6 mm×36/38/40 mm, wherein the end structures and wire density are programed into instructions and/or hand woven or machined by a STEEGER USA Model Number HS80/48-2021-IMC7K brand of machine in whole or in part.

6. The stent of claim 5, starting with at least stainless steel bands round a mandrel before hitting.

7. The stent of claim 6, having more density in the middle than the ends by having machine braids more consistently achieved efficiently at scale.

8. The stent of claim 7, being comprised of more density in the middle with flexure improved on the ends.

9. The stent of claim 8, having at least about 5/20/5 wire density machine made on 30 CM.

10. The stent of claim 9, having a more consistent machine braid imparted by the STEEGER USA Model Number HS80/48-2021-IMC7K brand of machine in whole or in part.

11. The stent of claim 10, configured to have carotid specific step down stent usages to deal with anatomic and pressure based challenges in the carotid artery.

12. A method of making a carotid stent, comprised of, in combination:
Providing a mandrel and having 2 bands around the mandrel before; winding/braiding/generating resultory stenting structures further comprising:
16 wires at 10 mm×40 mm, wherein the end structures and wire density are programed into instructions and/or hand woven or machined by a STEEGER USA Model Number HS80/48-2021-IMC7K brand of machine, in whole or in part.

13. A method of making a carotid stent, comprised of, in combination:
Providing a mandrel and having 2 bands around the mandrel before; winding/braiding/generating resultory stenting structures further comprising:
20 wires at 10 mm×40 mm, wherein the end structures and wire density are programed into instructions and/or hand woven or machined by a STEEGER USA Model Number HS80/48-2021-IMC7K brand of machine in whole or in part, further comprising laser welds, marker bands or coils and UV glue.
